(19) United States
(12) Patent Application Publication
(10) Pub. No.: US 2006/0014719 A1
(43) Pub. Date: Jan. 19, 2006

(54) Method for treating neurodegenerative diseases

(75) Inventor: Yan Chang, Ashland, MA (US)

Correspondence Address: FISH & NEAVE IP GROUP;ROPES & GRAY LLP, ONE INTERNATIONAL PLACE, BOSTON, MA, 02110-2624, US

(73) Assignee: GlycoGenesys, Inc., Boston, MA (US)

(21) Appl. No.: 11/181,094

(22) Filed: Jul. 13, 2005

Related U.S. Application Data
(60) Provisional application No. 60/587,398, filed on Jul. 13, 2004.

(51) Int. Cl. A61K 31/732

(52) U.S. Cl. 514/54

(57) ABSTRACT

The present invention is directed to methods and compositions for treatment of neurodegenerative diseases. In particular embodiments, the invention is directed to the treatment of Alzheimer's disease, MS, ALS, Huntington's Disease. In other embodiments, the invention is directed to the treatment of multiple sclerosis.

### CROSS-REFERENCE TO RELATED APPLICATIONS

This application claims the benefit of U.S. Provisional Application No. 60/587398 filed Jul. 13, 2004, entitled “Method for Treating Neurodegenerative Diseases,” the disclosure of which is incorporated herein in its entirety.

### BACKGROUND OF THE INVENTION

Alzheimer's disease (AD) is an etiologically indeterminate, non-infectious neurological disorder that shows progressive dementia. AD is the most common cause of dementia in the elderly, with about 3 to 5% of people over 65 suffering from AD.

While the definitive characteristic of AD is a postmortem observation of amyloid plaques and neurofibrillary tangles (malformations within nerve cells) in the brain of a patient, guidelines have been established to aid the diagnosis of AD in a living patient. Hallmarks of Alzheimer's disease include progressive nature of dementia, and degeneration of the cholinergic neurons of the basal forebrain. Characteristic positron emission tomography is observed, showing reduced 2FDG metabolism in parietal and temporal lobe association and posterior cingulate cortices, and in patients with advanced clinical symptoms, prefrontal association cortices, but usually not in primary sensory and motor cortical regions. Subcortical structures, including the basal ganglia, thalamus, brainstem and cerebellum, are preserved in typical AD. Additionally, increase in biomarkers such as total tau, and phosphorylated tau in the cerebrospinal fluid aids the diagnosis of Alzheimer's disease. For a recent review of biological markers of AD, see Frank, R. A. et al. (2003) Neurobiol. Aging 24:521-536, the disclosure of which is incorporated herein by reference in its entirety. Currently, there is no known cure for AD, and effectiveness of various existing treatments is limited.

Multiple sclerosis (MS) is the most common demyelinating disorder of the central nervous system, causing patches of sclerosis (i.e., plaques) in the brain and spinal cord. MS has protean clinical manifestations, depending upon the location and size of the plaque. Typical symptoms include visual loss, diplopia, nystagmus, dysarthria, weakness, paresthesias, bladder abnormalities, and mood alterations. Myriad treatments have been proposed for this long-term variable illness. The list of proposed treatments encompasses everything from diet to electrical stimulation to acupuncture, emotional support, and various forms of immunosuppressive therapy. None have proved to be satisfactory.

Amyotrophic lateral sclerosis (ALS), also known as Lou Gehrig's disease, is a progressive degeneration of motor neurons in the brain and spinal column. ALS causes increasing muscle weakness, inability to control movement, and problems with speaking, swallowing, and breathing. In the United States and most other parts of the world, 1 to 2 people per 100,000 develop ALS each year. In 5-10% of the patients, genetic components have been implicated. Men are affected slightly more often than women. Although ALS may occur at any age, it is most common in middle-aged and older adults. The etiology of the disease is unknown, but neuroinflammation has been recently suggested as a factor in pathogenesis of ALS.

There is no cure for ALS. In general, weakness progresses steadily without periods of improvement or stability. Eventually ALS leads to death, usually within 3 to 6 years. Treatment focuses on sustaining strength and independence and avoiding complications for as long as possible.

Huntington's Disease (HD) is a progressive, neurodegenerative disorder characterized by the gradual development of involuntary muscle movements affecting the hands, feet, face, and trunk and progressive deterioration of cognitive processes and memory (dementia). The affected neurons are generally in the basal ganglia and cerebral cortex regions of the brain. Neurologic movement abnormalities may include uncontrolled, irregular, rapid, jerky movements (chorea) and athetosis, a condition characterized by relatively slow, writhing involuntary movements. Dementia is typically associated with progressive disorientation and confusion, personality disintegration, impairment of memory control, restlessness, agitation, and other symptoms and findings. In individuals with the disorder, disease duration may range from approximately 10 to 25 years or more. Currently there are approximately 30,000 people in the United States afflicted with this condition. HD is a genetic disease, transmitted as an autosomal dominant trait. Components of inflammatory cascades, such as caspases, have been seen to take part in the manifestation of pathology of HD. There is no effective treatment for HD.

Evidence now suggests these pathological conditions have important inflammatory and immune components and may be amenable to treatment by anti-inflammatory and immunotherapeutic approaches. Weiner, H. L. and Selko, D. J., (2002) Nature 420: 879-884. In MS, episodic inflammation occurs in the initial stage of the disease, associated with discrete attacks of neurological dysfunction followed by recovery, which may leave residual neurological damage. Subsequently the disease often progresses to a stage where there is less inflammation and nervous system damage is caused by a degenerative process initiated by the inflammation. Id.

Unlike MS, the inflammation in AD seems to arise from inside the CNS with little or no involvement of lymphocytes or monocytes beyond their normal surveillance of the brain. Accumulation of beta amyloid (Aβ) leads to stimulation of the innate immune response, including activation of microglia and astrocytes, release of cytokines such as TNF-α and IL-β, complement activation and free-radical formation. This innate immune activation may contribute to neurotoxicity.

Accordingly, safe and effective therapies for neurodegenerative diseases such as MS, AD, ALS, and HD are needed.

### BRIEF SUMMARY OF THE INVENTION

One aspect of the invention provides methods for treating inflammatory neurodegenerative diseases such as Alzheimer's disease, amyotrophic lateral sclerosis, Huntington's disease or multiple sclerosis by administering a composition comprising a carbohydrate polymer.

Another aspect of the invention provides methods for prophylactic treatment for the prevention of inflammatory neurodegenerative diseases by administering to a subject at risk of developing such a disease a composition comprising a carbohydrate polymer.

Another aspect of the invention provides a kit that includes (i) a therapeutically effective amount of a carbohydrate polymer; and (ii) instructions and/or a label.

A preferred class of compound to be used in the method of the present invention comprises a carbohydrate with a polymeric backbone, optionally having side chains dependent therefrom. The side chains are terminated by a galactose, rhamnose, xylose, or arabinose unit. This material may be synthetic, natural, or semi-synthetic. In one particular embodiment, the therapeutic compound comprises a partially demethoxylated polygalacturonic acid backbone which may be interrupted with rhamnose residues. In another embodiment, the therapeutic compounds comprise homogalacturonan backbones with no pendent side chains. Such compounds may be prepared from naturally occurring pectin, and are referred to as partially depolymerized pectin or modified pectin. The most preferred class of carbohydrate for use in the present invention comprises a polygalacturonan backbone with side chains terminating in galactose.

The method of present invention may be administering such materials orally, by injection, transdermally, subcutaneously or by topical application, depending upon the specific type of neurodegenerative disorder being treated, and the adjunct therapy.

### DETAILED DESCRIPTION OF THE INVENTION

I. Overview

The present invention is directed to methods for augmenting treatment of inflammatory neurodegenerative diseases such as Alzheimer's disease or multiple sclerosis. The term “inflammatory neurodegenerative disease,” as used herein, refers to any pathological condition that manifests as a part of its etiology or symptom inflammation of neural tissues which results in degeneration and destruction of such neural tissues. Examples of inflammatory neurodegenerative disease include Alzheimer's disease (AD), multiple sclerosis (MS), Parkinson's disease (PD), amyotrophic lateral sclerosis (ALS), and Huntington's disease (HD).

Cell adhesion and activation of endothelial cell walls are important components of diseases such as MS or AD. The process involves a complex cascade of various molecules and mediators, including chemokines, adhesion molecules, and matrix metalloproteases. One important set of components is integrins, which may mediate the progression of these diseases.

Integrins are the principal receptors on animal cells for binding most extracellular matrix proteins, including collagen, fibronectin, and laminin. Integrins form a large family of homologous transmembrane linker proteins, and are the main way that cells bind to and respond to the extracellular matrix. Integrins are heterodimers of α and β subunits, and the ligand-binding site is composed of parts of both chains. In mammals, at least 22 integrin heterodimers, composed of 17 types of α subunits and 8 types of β subunits, are known. A single β chain can interact with multiple a chains, forming integrins that bind different ligands.

β integrins are generally believed to be directly associated with focal adhesion kinase (FAK). Constitutive activation of FAK is sufficient to rescue epithelial cells from anoikis, whereas apoptosis occurs if FAK is inhibited by, for example, a peptide representing the FAK-binding site of β1 integrin. Several proteins that lie downstream to β1 integrin in this signal transduction pathway have been identified, two of which are Akt and PI-3 kinase. Akt is a survival-promoting Ser-Thr protein kinase whose activity is regulated by a variety of growth factors in a PI 3-kinase-dependent manner. Activation of Akt has been shown to result in inhibition of apoptosis in several types of cells, including primary culture of cerebellar neurons, Rat-1, and COS-7 cells. P13 kinase and Akt both can be direct or indirect down stream effectors of FAK.

There is evidence that the carbohydrate polymer for the use in the method of present invention interacts directly or indirectly with integrins, more specifically β integrins, and prevents or attenuates the progression of the inflammatory neurodegenerative diseases. The carbohydrate useful to carry out the methods of the present invention has been shown to downregulate both FAK and Akt.

A preferred class of compound to be used in the method of the present invention comprises a carbohydrate with a polymeric backbone, optionally having side chains dependent therefrom. Such side chains are preferably terminated by a galactose, rhamnose, xylose, or arabinose unit. This material may be synthetic, natural, or semi-synthetic. In one particular embodiment, the therapeutic compound comprises a partially demethoxylated polygalacturonic acid backbone which may be interrupted with rhamnose residues. In another embodiment, the therapeutic compounds comprise homogalacturonan backbones with no pendent side chains. Such compounds may be prepared from naturally occurring pectin, and are referred to as partially depolymerized pectin or modified pectin. These molecules are more fully described below.

The present invention provides methods to treat an inflammatory neurodegenerative disease by administering a carbohydrate polymer to a patient afflicted with such a disease. The present invention also provides methods for a prophylactic treatment of an inflammatory neurodegenerative disease by administering a carbohydrate polymer to a subject at risk of developing such a disease.

II. Definitions

As used herein, the terms “agent” and “compound” include both protein and non-protein moieties. An agent may be a small organic molecule, a polypeptide, a protein, a peptide complex, a peptidomimetic, a non-peptidyl agent, or a polynucleotide.

As used herein, “ameliorates” means alleviate, lessen, or and decrease the extent of a symptom or decrease the number of occurrence of episodes of a disease manifestation.

As used herein the term “animal” refers to mammals, preferably mammals such as humans. Likewise, a “patient” or “subject” to be treated by the method of the invention can mean either a human or non-human animal.

The terms “apoptosis” or “programmed cell death,” refers to the physiological process by which unwanted or useless cells are eliminated during development and other normal biological processes. Apoptosis is a mode of cell death that occurs under normal physiological conditions and in which the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy. Cells undergoing apoptosis show characteristic morphological and biochemical features. These features include chromatin aggregation, nuclear and cytoplasmic condensation, partition of cytoplasm and nucleus into membrane bound vesicles (apoptotic bodies) which contain ribosomes, morphologically intact mitochondria and nuclear material. Cytochrome C release from mitochondria is seen as an indication of mitochondrial dysfunction accompanying apoptosis. In vivo, these apoptotic bodies are rapidly recognized and phagocytized by either macrophages or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis.”

The term “preventing” is art-recognized, and when used in relation to a condition, such as recurrence or onset of a disease such as multiple sclerosis (MS) or Alzheimer's disease (AD), a syndrome complex such as dementia or any other medical condition, is well understood in the art, and includes administration of a treatment which reduces the frequency of, or delays the onset of, symptoms of a medical condition in a subject relative to a subject which does not receive the treatment. Thus, prevention of AD or MS includes, for example, delaying the onset of the disease or stopping progression of the disease beyond the early stage in a treated population compared to untreated population. Prevention of symptoms of neurodegenerative diseases such as motor impairment or vision loss includes, for example, slowing the progression of loss of function or delaying the appearance of such loss of function in a population of patients receiving the prophylactic treatment relative to an untreated control population. Prevention of symptoms of neurodegenerative diseases such as memory impairment or deficiency in cognitive functions, includes, for example, reducing the number of episodes of failed recollection or cognitive impairment in a population of patients receiving a prophylactic treatment relative to an untreated control population, and/or delaying the appearance of memory deficiency in a treated population versus an untreated control population, e.g., by a statistically and/or clinically significant amount.

“Treat”, as used herein, means at least lessening the severity or ameliorating the effects of, for example, one or more symptoms, of a disorder or condition.

III. Exemplary Embodiments

A. Materials Useful to Practice Present Invention

One class of compounds contemplated by the present invention is carbohydrate-containing polymers. Materials useful in the present inventions may be generally comprised of natural or synthetic polymers and oligomers. Preferably, such polymers are very low in toxicity.

A preferred class of polymers for the practice of the present invention are carbohydrate-derived polymers, comprising oligomeric or polymeric species of natural or synthetic origin, rich in galactose or arabinose. Such materials will preferably have a molecular weight in the range of up to 500,000 daltons (Da) and, more preferably, in the range of up to 150,000 Da. One particular material comprises a partially demethoxylated polygalacturonic acid backbone which may be interrupted by rhamnose with galactose-terminated side chains pendent therefrom. Another particular material comprises a homogalacturonan backbone with or without side chains pendent therefrom.

One group of materials falling within this general class comprises a partially demethoxylated polygalacturonic acid backbone having rhamnose, galactose, arabinose or other sugar residues pendent therefrom. In certain embodiments, modified pectins useful to practice the invention are described by formulae I and II below, and it is to be understood that yet other variants of this general compound may be prepared and utilized in accord with the principles of the present invention.

1. Homogalacturonan
-[α-D-GalpA-(1→4)-α-D-GalpA]ₙ- (I)

2. Rhamnogalacturonan

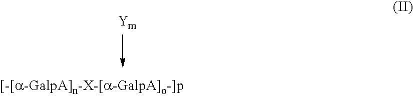

In the formulae above, m is ≧0, n, o and p are ≧1, X is α-Rhap; and Ym represents a linear or branched chain of sugars (each Y in the chain Ym can independently represent a different sugar within the chain). The sugar Y may be, but is not limited to, any of the following: α-Galp, β-Galp, β-Apif, β-Rhap, α-Rhap, α-Fucp, β-GlcpA, α-GalpA, β-GalpA, β-DhapA, Kdop, β-Acef, α-Araf, β-Araf, and α-Xylp. Ym may be

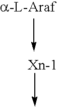

Abbreviated sugar monomer names used herein are defined as follows: GalA: galacturonic acid; Rha: rhamnose; Gal: galactose; GlcA: glucuronic acid; DhaA: 3-deoxy-D-lyxo-heptulosaric acid; Kdo: 3-deoxy-D-manno-2-octulosonic acid; Ace: aceric acid (3-C-carboxy-5-deoxy-L-lyxose); Ara: arabinose. Italicized p indicates the pyranose form, and italicized f indicates a furanose ring.

Another class of compound useful for the present invention are represented by formulae III and IV. 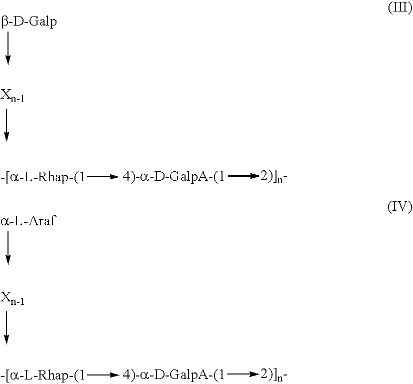
In the above representations, n is an integer greater than 1, Xₙ₋₁ represents a short side-chain of neutral sugar residues, X can be any of several sugars found in pectin side chains, including but not limited to β-Apif, β-Rhap, α-Fucp, β-GlcpA, α-GalpA, β-GalpA, β-DhapA, Kdop, β-Acef, α-Galp, and α-Araf.

It will be understood that natural pectin does not possess a strictly regular repeating structure, and that additional random variations are likely to be introduced by partial hydrolysis of the pectin, so that the identity of Ym and the values of n and o may vary from one iteration to the next of the p repeating units represented by formula II above.

An exemplary polymer of this type is modified pectin, preferably water soluble pH modified citrus pectin. Suitable polymers of this type are disclosed in, for example U.S. Pat. Nos. 5,834,442, 5,895,784, 6,274,566 and 6,500,807, and PCT Publication WO 03/000,118.

Pectin is a major constituent of plant cell walls, and is a combination of at least three principal pectic polysaccharides, which are believed to be covalently linked within the cell wall: homogalacturonan (HG), rhamnogalacturonan I (RG-I), and rhamnogalacturonan II (RG-II).

HG is a linear homopolymer of 1,4-linked α-D-galacturonic acid, methyl esterified to varying degrees at C-6. Depending on the species of plant, the backbone galacturonic acid units may be C-3 substituted with O-acetyl residues.

RG-I is a heterologous group of polysaccharides that contain a backbone of the repeating disaccharide [→4)-α-D-GalpA-(1→2)-α-L-Rhap-(1→]. Between 20 and 80% of the Rhap residues are substituted at C-4 with neutral oligosaccharide side chains containing linear and branched α-L-Araf and β-D-Galp residues. The backbone GalpA residues of RG-I are not typically substituted with polysaccharides, although they may be O-acetylated at C2 or C3. Herein Fucp is fucose, GlcpA is glucuronic acid.

RG-II has a more highly conserved structure, with a backbone usually composed of at least seven to nine 1,4-linked α-D-GalpA residues, to which four complex oligosaccharide side chains are typically attached at C-2 and/or C-3.

Pectin itself is thought to be a heteropolysaccharide with a backbone composed of alternating HG (“smooth regions”) and RG (“hairy regions”). The smooth regions are linear polymers of 1,4-linked α-D-galacturonic acid.

The highly branched “hairy regions” feature neutral sugar units (typically D-galactose or L-arabinose or xylose attached by glycosidic linkages to the C4 atoms of the rhamnose units, and/or to the C2 or C3 atoms of the galacturonic acid units. Depending upon the extraction process used, the hairy regions are partially or largely degraded during the manufacture of commercial pectin, leaving intact the smooth polygalacturonic acid regions, with a smaller number of neutral sugar units still attached to or embedded in the main linear chain. The methyl galacturonate ester groups survive the extraction process, although degree of methyl esterification may be reduced in subsequent processing steps to provide commercial pectins having various utilities.

The degree of methyl esterification in most commercial pectins varies from 0-90%. If 50% or more of the carboxyl groups are esterified the pectin is referred to as a “high ester” or “high methoxyl” pectin“. If less than 50% of the carboxyl groups are esterified then the pectin is referred to as a “low ester” or “low methoxyl” pectin. Pectin having few or no esterified groups is referred to as pectic acid.

The choice of the starting pectin material affects the characteristics of the final product. However, in choosing a starting pectin, important things to consider are molecular weight, degree of esterification, monosaccharide content, linkage, polydispersity and so forth. The starting pectin often contains less than 10% methyl ester by total mass, has a molecular weight of greater than 150 kD, and has a particular monosaccharide content, e.g., galactose content, greater than or equal to 5%. In one embodiment of the invention the starting pectin composition may comprise approximately equal amounts of HG and RG-I, preferably at least 70% HG and less than 30% RG-I more preferably at least 80% HG and less than 20% RG-I and most preferably at least 90% HG and less than 10% RG-I. In certain embodiments, the pectin may contain 0-10% of RG-II. In certain preferred embodiments, the starting pectin is citrus pectin.

U.S. Pat. No. 5,895,784, the disclosure of which is incorporated herein by reference, describes modified pectin materials, techniques for their preparation, and use of the material as a treatment for various cancers. The material of the '784 patent is described as being prepared by a pH based modification procedure in which the pectin is put into solution and exposed to a series of programmed changes in pH which results in the breakdown of the molecule to yield therapeutically effective modified pectin. The material in the '784 patent is most preferably prepared from citrus pectin; although, it is to be understood that modified pectins may be prepared from pectin from other sources, such as apple pectin. Also, modification may be done by enzymatic treatment of the pectin, or by physical processes such as heating. Further disclosure of modified pectins and techniques for their preparation and use are also found in U.S. Pat. No. 5,834,442, U.S. patent application Ser. No. 08/024,487, and U.S. patent application Ser. No. 11/093,268, the disclosures of which are incorporated herein by reference. Modified pectins of this type generally have molecular weights in the range of less than 100 kDa. A group of such materials has an average molecular weight of less than 3 kDa. Another group has an average molecular weight in the range of 1-15 kDa, with a specific group of materials having an average molecular weight of about 10 kDa. In one embodiment, modified pectin has the structure of a pectic acid polymer with some of the pectic side chains still present. In preferred embodiments, the modified pectin is a copolymer of homogalacturonic acid and rhamnogalacturonan I in which some of the galactose- and arabinose-containing sidechains are still attached. More preferred embodiment of the present invention is a modified pectin composition that comprises or consists essentially of a homogalacturonan backbone with small amounts of rhamnogalacturonan interspersed therein, with neutral sugar side chains, and has a low degree of neutral sugar branching dependent from the backbone. In certain embodiments, the modified pectin is partially depolymerized, so as to have a disrupted homogalacturonan backbone. The modified pectin may have a molecular weight of 1 to 500 kDa, preferably 10 to 250 kDa, more preferably 50-200 kDa, more preferably 70-150 kDa, even more preferably 80-150 kDa, and most preferably 80 to 100 kDa as measured by Gel Permeation Chromatography (GPC) with Multi Angle Laser Light Scattering (MALLS) detection.

Degree of esterification is another characteristic of modified pectins. Naturally occurring pectins are methoxylated so that the methoxyl groups account for up to 10% of the total mass of the pectin. Degree of methoxylation is a variable that affect the biological and pharmacological activities of modified pectin. Modified pectins are demethoxylated to various degrees and contain reduced amounts of methoxyl groups compared to naturally occurring pectins.

Saccharide content is another characteristic of modified pectins. In certain embodiments, the modified pectin is composed entirely of a single type of saccharide subunit. In other embodiments, the modified pectin comprises at least two, preferably at least three, and most preferably at least four types of saccharide subunits. For example, the modified pectin may be composed entirely of galacturonic acid subunits. Alternatively, the modified pectin may comprise a combination of galacturonic acid and rhamnose subunits. In yet another example, the modified pectin may comprise a combination of galacturonic acid, rhamnose, and galactose subunits. In yet another example, the modified pectin may comprise a combination of galacturonic acid, rhamnose, and arabinose subunits. In still yet another example, the modified pectin may comprise a combination of galacturonic acid, rhamnose, galactose, and arabinose subunits. In some embodiments, the galacturonic acid content of modified pectin is greater than 50%, preferably greater than 60% and most preferably greater than 80%. In some embodiments, the rhamnose content is less than 25%, preferably less than 15% and most preferably less than 10%; the galactose content is less than 50%, preferably less than 40% and most preferably less than 30%; and the arabinose content is less than 15%, preferably less than 10% and most preferably less than 5%. In certain embodiments, the modified pectin may contain other uronic acids, xylose, ribose, lyxose, glucose, allose, altrose, idose, talose, gluose, mannose, fructose, psicose, sorbose or talalose in addition to the saccharide units mentioned above.

Modified pectin suitable for use in the subject methods may also have any of a variety of linkages or a combination thereof. By linkages it is meant the sites at which the individual sugars in pectin are attached to one another. In some embodiments, the modified pectin comprises only a single type of linkage. In certain preferred embodiments, the modified pectin comprises at least two types of linkages, and most preferably at least 3 types of linkages. For example, the modified pectin may comprise only alpha-1,4-linked galacturonic acid subunits. Alternatively, the modified pectin may comprise alpha-1,4-linked galacturonic acid subunits and alpha-1,2-rhamnose subunits. In another example, the modified pectin may be composed of alpha-1,4-linked galacturonic acid subunits and alpha-1,2-rhamnose subunits linked through the 4 position to arabinose subunits. In another example, the modified pectin may comprise alpha-1,4-linked galacturonic acid subunits and alpha-1,2-rhamnose subunits linked through the 4 position to arabinose subunits with additional 3-linked arabinose subunits. In another example, the modified pectin may comprise alpha-1,4-linked galacturonic acid subunits and alpha-1,2-rhamnose subunits linked through the 4 position to arabinose subunits with additional 5-linked arabinose units. In another example, the modified pectin may comprise alpha-1,4-linked galacturonic acid subunits and alpha-1,2-rhamnose subunits linked through the 4 position to arabinose subunits with additional 3-linked and 5-linked arabinose subunits. In another example, the modified pectin may comprise alpha-1,4-linked galacturonic acid subunits and alpha-1,2-rhamnose subunits linked through the 4 position to arabinose subunits with additional 3-linked and 5-linked arabinose subunits with 3,5-linked arabinose branch points. In another example, the modified pectin may comprise alpha-1,4-linked galacturonic acid subunits and alpha-1,2-rhamnose subunits linked through the 4 position to galactose subunits. In another example, the modified pectin may comprise alpha-1,4-linked galacturonic acid subunits and alpha-1,2-rhamnose subunits linked through the 4 position to galactose subunits with additional 3-linked galactose subunits. In another example, the modified pectin may comprise alpha-1,4-linked galacturonic acid subunits and alpha-1,2-rhamnose subunits linked through the 4 position to galactose subunits with additional 4-linked galactose subunits. In another example, the modified pectin may comprise alpha-1,4-linked galacturonic acid subunits and alpha-1,2-rhamnose subunits linked through the 4 position to galactose subunits with additional 3-linked galactose subunits with 3,6-linked branch points. In another example, the modified pectin may comprise alpha-1,4-linked galacturonic acid subunits and alpha-1,2-rhamnose subunits linked through the 4 position to galactose subunits with additional 4-linked galactose subunits with 4,6-linked branch points. In certain embodiments, the side chains of the modified pectin may comprise uronic acids, galacturonic acid, glucuronic acid, rhamnose, xylose, ribose, lyxose, glucose, allose, altrose, idose, talose, gluose, mannose, fructose, psicose, sorbose or talalose in addition to the saccharide units described above.

In certain embodiments, the modified pectin preparation is a substantially ethanol-free product suitable for parenteral administration. By substantially free of ethanol, it is meant that the compositions of the invention contain less than 5% ethanol by weight. In preferred embodiments the compositions contain less than 2%, and more preferably less than 0.5% ethanol by weight. In certain embodiments, the compositions further comprise one or more pharmaceutically acceptable excipients. Such compositions include aqueous solutions of the modified pectin of the invention. In certain embodiments of such aqueous solutions, the pectin modification occurs at a concentration of at least 7 mg/mL, and preferably at least 10 or even 15 or more mg/ml. Any of such compositions are also substantially free of organic solvents other than ethanol.

Yet another class of compound useful to carry out the methods of present invention is galactomannan. Galactomannan is a polysaccharide comprising mannose backbone with galactose pendent therefrom. Galactomannan is found in nature and can be isolated from plant materials as well as from yeasts, having molecular weight in the range of 20-600 kDa, 90-415 kDa or 40-200 kDa depending on the source. In specific examples, the galactomannan may have an average molecular weight of 50, 83, or 215 kDa. In a preferred embodiment, the galactomannan may be a β-1,4 D-galactomannan. Moreover, the galactomannan may include a ratio of 2.0-3.0 mannose to 0.5-1.5 galactose. The ratio of mannose to galactose may be about 1.13 mannose to 1 galactose, 1.7 mannose to 1 galactose, 2.6 mannose to 1.5 galactose, or 2.2 mannose to 1 galactose.

B. Administration

The method of present invention may be used to treat neurodegenerative diseases such as Alzheimer's disease, multiple sclerosis, Parkinson's disease, amyotrophic lateral sclerosis, or Huntington's disease.

Another embodiment of the present invention is prophylactic treatment of a subject at risk of developing a neurodegenerative disease. A subject at risk is identified by, for example, determining the genetic susceptibility to a known neurodegenerative disease based on family history or genetic markers.

A compound suitable for the practice of the present invention may be administered orally, parenterally by intravenous injection, transdermally, intrathecally, by pulmonary inhalation, by intravaginal or intrarectal insertion, by subcutaneous implantation, intramuscular injection.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; for oral dosage, a syrup, tablet or palatable solution; for administration by inhalation, a microcrystalline powder or a solution suitable for nebulization; for intravaginal or intrarectal administration, pessaries, suppositories, creams or foams. Preferably, the route of administration is parenteral, more preferably intravenous.

In general, an embodiment of the invention is to administer a suitable daily dose of a therapeutic composition that will be the lowest effective dose to produce a therapeutic effect, for example, mitigating symptom. The therapeutic carbohydrates are preferably administered at a dose per subject per day of at least about 2 mg, at least about 5 mg, at least about 10 mg, or at least about 20 mg as appropriate minimal starting dosages. In one embodiment of the methods described herein, a dose of about 0.01 to about 500 mg/kg can be administered. In general, the effective dosage of the compound in the present invention is about 50 to about 400 micrograms of the compound per kilogram of the subject per day. However, it is understood by one skilled in the art that the dose of the composition to practice the invention will vary depending on the subject and upon the particular route of administration used. It is routine in the art to adjust the dosage to suit the individual subjects. Additionally, the effective amount may be based upon, among other things, the size of the compound, the biodegradability of the compound, the bioactivity of the compound and the bioavailability of the compound. If the compound does not degrade quickly, is bioavailable and highly active, a smaller amount will be required to be effective. The actual dosage suitable for a subject can easily be determined as a routine practice by one skilled in the art, for example a physician or a veterinarian given a general starting point.

The compound may be delivered hourly, daily, weekly, monthly, yearly (e.g., in a time release form) or as a one-time delivery. The delivery may be continuous delivery for a period of time, e.g., intravenous delivery. In one embodiment of the methods described herein, the therapeutic composition is administered at least once per day. In one embodiment, the therapeutic composition is administered daily. In one embodiment, the therapeutic composition is administered every other day. In one embodiment, the therapeutic composition is administered every 6 to 8 days, or more specifically, weekly.

In one embodiment of the methods described herein, the route of administration can be oral, intraperitoneal, transdermal, subcutaneous, by intravenous or intramuscular injection, by inhalation, topical, intralesional, infusion; liposome-mediated delivery; intrathecal, gingival pocket, rectal, intrabronchial, nasal, transmucosal, intestinal, ocular or otic delivery, or any other methods known in the art as one skilled in the art may easily perceive. In other embodiments of the invention, the compositions incorporate particulate forms protective coatings, hydrolase inhibitors or permeation enhancers for various routes of administration, including parenteral, pulmonary, nasal and oral.

An embodiment of the method of present invention is to administer the carbohydrate polymer describes herein in a sustained release form. Such method comprises implanting a sustained-release capsule or a coated implantable medical device so that a therapeutically effective dose of the carbohydrate polymer is continuously delivered to a subject of such a method. The carbohydrate polymer may be delivered via a capsule which allows sustained-release of the agent or the peptide over a period of time. Controlled or sustained-release compositions include formulation in lipophilic depots (e.g., fatty acids, waxes, oils). Also comprehended by the invention are particulate compositions coated with polymers (e.g., poloxamers or poloxamines).

C. Examples

The foregoing discussion and description is illustrative of specific embodiments, but is not meant to be a limitation upon the practice thereof. It is the following claims, including all equivalents, which define the scope of the invention.

We claim:

1. A method for treating a patient afflicted with a neurodegenerative disease, comprising administering to said patient a pharmaceutical composition comprising a therapeutically effective amount of a carbohydrate having a partially demethoxylated polygalacturonic acid backbone which may be interrupted by rhamnose.

2. A method for a prophylactic treatment of a subject at risk of developing a neurodegenerative disease, comprising administering to said patient a pharmaceutical composition comprising a therapeutically effective amount of a carbohydrate having a partially demethoxylated polygalacturonic acid backbone which may be interrupted by rhamnose.

3. A method for treating a patient afflicted with a neurodegenerative disease, comprising administering to said patient a pharmaceutical composition comprising a therapeutically effective amount of a carbohydrate that binds to an integrin.

4. The method of claim 1, 2, or 3, wherein said neurodegenerative disease is selected from Alzheimer's disease, multiple sclerosis, amyotrophic lateral sclerosis, or Huntington's disease.

5. The method of any of claims 1 to 4, wherein said carbohydrate comprises a partially demethoxylated polygalacturonic acid interrupted by rhamnose residues.

6. The method of any of claims 1 to 4, wherein said backbone comprises homogalacturonan.

7. The method of any of claims 1 to 4, wherein said carbohydrate further comprises neutral sugar side chains having a low degree of branching dependent from the backbone.

8. The method of any one of claims 1 to 4, wherein said carbohydrate is a partially depolymerized pectin.

9. The method of claim 8, wherein said partially depolymerized pectin comprises a pH modified pectin, an enzymatically modified pectin, and/or a thermally modified pectin.

10. The method of claim 8, wherein said partially depolymerized pectin comprises a modified citrus pectin.

11. The method of claim 8, wherein said partially depolymerized pectin has a molecular weight of 1 to 500 kilodaltons (kDa).

12. The method of claim 8 wherein said partially depolymerized pectin has a molecular weight of 10 to 250 kDa.

13. The method of claim 8 wherein said partially depolymerized pectin has a molecular weight of 50 to 200 kDa.

14. The method of claim 8 wherein said partially depolymerized pectin has a molecular weight of 70 to 150 kDa.

15. The method of claim 8 wherein said partially depolymerized pectin has a molecular weight of 80 to 100 kDa.

16. The method of any one of claims 1 to 4, wherein said composition is administered parenterally.

17. The method of claim 16, wherein said composition is administered by intravenous infusion.

18. The method of any one of claims 1 to 4, wherein said composition is administered orally.

19. The method of any one of claims 1 to 4, wherein said composition is administered by inhalation.

20. The method of any one of claims 1 to 4, wherein said composition is administered by subcutaneous injection.

21. The method of any one of claims 1 to 4, wherein said composition is administered by intramuscular or intraperitoneal injection or infusion.

22. The method of any one of claims 1 to 4, wherein said composition is administered intrathecally.

23. A kit comprising (i) a pharmaceutical composition comprising a therapeutically effective amount of carbohydrate with a partially demethoxylated polygalacturonic acid backbone; and (ii) instructions and/or a label for administration of the composition for the treatment of patients afflicted with a neurodegenerative disease.
